(19) United States
(12) Patent Application Publication
(10) Pub. No.: US 2024/0245765 A1
(43) Pub. Date: Jul. 25, 2024

(54) Use of Ectromelia Virus for Cancer Immunotherapy and Vaccines

(71) Applicants: Thomas Jefferson University, Philadelphia, PA (US); POXIMMUNOMICS LLC, Gaithersburg, MD (US)

(72) Inventors: Renhuan Xu, Gaithersburg, MD (US); Luis Javier Sigal, Glenside, PA (US); Cory Knudson, Philadelphia, PA (US); Linjuan Tang, Philadelphia, PA (US)

(21) Appl. No.: 18/560,538

(22) PCT Filed: May 13, 2022
(86) PCT No.: PCT/US22/29158

§ 371 Date: Nov. 13, 2023

Related U.S. Application Data
(60) Provisional application No. 63/188,021, filed on May 13, 2021.

(51) Int. Cl. A61K 39/215; A61K 39/00; A61P 31/14; A61P 35/00; C12N 7/00; C12N 15/86

(52) U.S. Cl. CPC A61K 39/215; A61K 39/0011; A61P 31/14; A61P 35/00; C12N 7/00; C12N 15/86; A61K 2039/5254; C12N 2710/24022; C12N 2710/24034; C12N 2710/24043; C12N 2770/20034

(57) ABSTRACT

The invention provides an ectromelia virus vector for expression of heterologous sequences under the control of a viral early/late promoter, and methods of use thereof for immunotherapy, cancer treatment and treatment of infectious disease.

### CROSS REFERENCE TO RELATED APPLICATIONS

This application claims priority to U.S. Provisional Application No. 63/188,021, filed May 13, 2021 which is hereby incorporated by reference herein in its entirety.

### BACKGROUND OF THE INVENTION

Orthopoxviruses (OPVs) are ˜200 Kb dsDNA viruses that are easy to modify genetically by homologous recombination. Due to their large size and complex genome, OPVs accept large DNA inserts without affecting their infectivity and replication. The most studied OPV is vaccinia virus (VACV), which is the smallpox vaccine. The VACV vaccine has been used for centuries, and its production can be scaled-up to hundreds of millions of doses. VACV can productively infect many species, including mice and humans. Indeed, in immunocompromised people, VACV can produce severe disease and death. Therefore, wild type (WT) VACV is not an ideal option as a vaccine vector.

The mouse-specific OPV ectromelia virus (ECTV) has a very narrow host specificity for the mouse. Therefore, it is apathogenic in all non-mouse species tested, such as rats, rabbits, guinea pigs, and hamsters (Burnet et al., 1946, Journal of immunology, 53:1-13; Flynn et al., 1963, Anl-6723. ANL Rep. 50-2; Flynn et al., 1962, Proc Anim Care Panel, 12:263-6). ECTV is also apathogenic in humans. Because of this, ECTV is classified as a biosafety level 1 (BSL1) pathogen. Despite being used in research for almost a century, there has never been a human infection report with ECTV. Over the course of approximately 17 years, many tools have been developed to make recombinant ECTV expressing a variety of proteins. Initially, the gene for green fluorescent protein (GFP) was introduced as a replacement for other genes that were to be targeted for deletion, such as the Type I interferon (IFN-I) decoy receptor EVM166 encoded by ECTV. The resulting virus, ECTV-Δ166, is attenuated in mice >7 orders of magnitude (4). GFP was also introduced in a noncoding region of the genome (Xu et al., 2008, The Journal of experimental medicine, 205(4):981-92). In subsequent studies, ECTV-Δ036 was generated, a mutant virus lacking a protein called EVM036, which is required for ECTV to spread from cell to cell in tissue culture. ECTV-4036 does not replicate well in tissue culture, forming tiny plaques and is extremely attenuated in vivo. Furthermore, a plasmid was produced whereby one could reintroduce EVM036 together with any protein of interest into ECTV-4036 to produce a new virus that generates the protein of interest and replicates normally (Roscoe et al., 2012, Journal of virology, 86(24): 13501-7). Therefore, a method of easily producing ECTV expressing any protein of interest has been created. Severe acute respiratory syndrome-coronavirus 2 (SARS-CoV-2) is the causative agent of Coronavirus (CoV) disease 2019 (COVID-19) (Zhu et al., 2020, N Engl J Med, 382(8): 727-33; Zhou et al., 2020, Nature, 579(7798):270-3). Following SARS-CoV-2 infection, people can remain asymptomatic or develop overt signs of disease, ranging from relatively minor discomfort of the upper respiratory tract to pneumonia that frequently develops into fatal acute respiratory distress syndrome (ARDS) (Zhou et al., 2020, Nature, 579(7798):270-3; Huang et al., 2020, Lancet, 395(10223):497-506). ARDS was also the main reason for death after infection with the highly related coronavirus SARS-CoV-1, which caused an epidemic in 2002-2003 (Hui et al., 2010, Infect Dis Clin North Am, 24(3):619-38; Rainer et al., 2004, Curr Opin Pulm Med, 10(3):159-65). The pathogenesis and immunobiology of SARS-CoV-1 and SARS-CoV-2 appear similar. Likely, this is the result of their genetic relatedness (˜79%) (Zhou et al., 2020, Nature, 579(7798):270-3; Wang et al., 2020, Eur J Clin Microbiol Infect Dis, 39(9): 1629-1635; Lu et al., 2020, Lancet, 395(10224):565-74), their use of human angiotensin-converting enzyme 2 (hACE2) as the receptor to enter cells (Wrapp et al., 2020, Science, 367(6483): 1260-3; Walls et al., 2020, Cell, 181(2):281-292.e6). The initial phase of SARS-CoV-1 and SARSCoV-2 pneumonia includes diffuse alveolar damage (DAD), characterized by protein-rich edema, inflammation, surfactant dysfunction, and severe hypoxia (Hui et al., 2010, Infect Dis Clin North Am, 24(3):619-38; Rainer et al., 2004, Curr Opin Pulm Med, 10(3):159-65). From there, DAD can progress into ARDS with pulmonary fibrosis, hyaline membrane formation, and eventually microangiopathy, angiogenesis, and thrombosis, followed by widespread organ failure (Rainer et al., 2004, Curr Opin Pulm Med, 10(3):159-65; Ackermann et al., 2020, N Engl J Med, 383(2): 120-128). Fatal disease and death from SARS-CoV-1 and -2 are more common in the aged, in males, and in people with co-morbidities such as heart disease and diabetes (Chen et al., 2020, Lancet, 395(10223):507-13; Wu et al., 2020, Nat Med. 2020; 26(4):506-10). However, not all aged individuals or those with co-morbidities develop severe disease (Chen et al., 2020, Lancet, 395(10223):507-13; Wu et al., 2020, Nat Med. 2020; 26(4):506-10). Furthermore, complications and death can also occur in apparently healthy young people (Liu et al., 2020, J Infect, 80(6):e14-e18). Critically, there are not yet ways to predict, prevent, or specifically treat COVID19 (Velavan et al., 2020, Int J Infect Dis, 95:304-7; Confalonieri et al., 2017, Eur Respir Rev, 26(144):160116), and a vaccine is sorely needed. Covid vaccines that are currently approved use adenovirus vectors (AV) (Kaur et al., 2020, Virus Res, 288:198114). If the immunity AV induce is short-lived, it is likely AV will not be useful for re-immunization due to immunity to the vector itself. Moreover, anti-AV immunity would prevent AV re-use for vaccines against other emerging viruses. Therefore, it is crucial to introduce novel vectors to the immune-modulating arsenal for COVID-19, cancer and other diseases.

Thus, there is a need in the art for improved compositions and methods for cancer immunotherapy and vaccination against viral infection. This invention satisfies this unmet need.

### SUMMARY OF THE INVENTION

In one embodiment, the invention relates to a recombinant ectromelia virus (ECTV) vector comprising at least one expression unit for expression of at least one heterologous nucleic acid sequence. In one embodiment, the at least one expression unit is under the control of an ECTV early/late promoter. In one embodiment, the ECTV vector is attenuated. In one embodiment, the early/late promoter is selected from the group consisting of 7.5 and H5.

In one embodiment, the ECTV vector comprises a deletion or inactivation of at least one immune evasion gene. In one embodiment, the at least one immune evasion gene is selected from the group consisting of a cytokine receptor homologue and a cytokine mimic.

In one embodiment, the ECTV vector further comprises one or more additional heterologous nucleotide sequence. In one embodiment, the one or more additional heterologous nucleotide sequence is a sequence encoding a therapeutic agent, a sequence encoding a targeting moiety, a sequence encoding a detectable and/or selectable marker, a pro-apoptotic gene, or a pro-necroptotic gene.

In one embodiment, the target nucleotide sequence encodes an antigenic polypeptide sequence, an antibody or an antibody fragment. In one embodiment, the antigenic polypeptide sequence is a cancer/tumor antigen, an autoantigen, an allergen, an antigen associated with hypersensitivity, a prion antigen, a viral antigen, a bacterial antigen, an antigen from protozoa or fungi, or a parasitic antigen.

In one embodiment, the at least one antigen comprises a cancer specific antigen.

In one embodiment, the at least one antigen comprises a viral antigen. In one embodiment, the viral antigen is selected from the group consisting of SARS-CoV-2 spike antigen, and a fragment of the SARS-CoV-2 spike antigen comprising the receptor binding domain (RBD).

In one embodiment, the at least one antigen is a bacterial antigen.

In one embodiment, the expression unit comprises at least two nucleotide sequences encoding antigenic polypeptides. In one embodiment, the at least two antigenic polypeptides are from two different viruses or from two different clades of the same virus.

In one embodiment, the expression unit comprises at least one antigenic polypeptide from a virus and at least one cancer-specific antigenic polypeptide.

In one embodiment, at least one antigen is a CTL-recognized epitope, a T helper cell-recognized epitope, or a B cell-recognized epitope.

In one embodiment, the invention relates to a vaccine comprising a recombinant ectromelia virus (ECTV) vector comprising at least one expression unit for expression of at least one heterologous nucleic acid sequence. In one embodiment, the at least one expression unit is under the control of an ECTV early/late promoter. In one embodiment, the ECTV vector is attenuated. In one embodiment, the early/late promoter is selected from the group consisting of 7.5 and H5.

In one embodiment, the composition further comprises a pharmaceutical carrier.

In one embodiment, the invention relates to a method for inducing an immune response in a subject comprising administering a vaccine comprising a recombinant ectromelia virus (ECTV) vector comprising at least one expression unit for expression of at least one heterologous nucleic acid sequence to the subject in an amount effective to induce an immune response. In one embodiment, the immune response comprises one or more of: the production of memory CD8+ T cells specific for the at least one antigen, the production of memory CD4+ T cells specific for the at least one antigen, and the production of antibodies specific for the at least one antigen.

In one embodiment, the invention relates to a method for treating cancer in a subject in need thereof, the method comprising administering a vaccine comprising a recombinant ectromelia virus (ECTV) vector comprising at least one expression unit for expression of at least one heterologous nucleic acid sequence to the subject.

In one embodiment, the recombinant ECTV vector comprises a target nucleotide sequence encoding a cancer antigen.

In one embodiment, the recombinant ECTV vector further comprises a target nucleotide sequence encoding a viral antigen. In one embodiment, the viral antigen is selected from the group consisting of an influenza viral antigen and a human cytomegalovirus antigen.

In one embodiment, the recombinant ECTV vector comprises a target nucleotide sequence encoding an immunotherapeutic antibody for the treatment of cancer.

In one embodiment, the invention relates to a method for treating a viral infection, or a disease or disorder associated therewith, in a subject in need thereof, the method comprising administering a vaccine comprising a recombinant ectromelia virus (ECTV) vector comprising at least one expression unit for expression of at least one heterologous nucleic acid sequence to the subject. In one embodiment, the recombinant ECTV vector comprises a target nucleotide sequence encoding a viral antigen. In one embodiment, the viral infection comprises SARS-CoV-2 infection. In one embodiment, the recombinant ECTV vector comprises a target nucleotide sequence encodes an immunotherapeutic antibody for the treatment of the viral infection.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIGS. 1A-B, depicts exemplary results demonstrating that ECTV-Luciferase (Luc) replicates locally at the site of infection in rats and induces anti-orthopoxvirus (OPV) and anti-Luc antibody responses. FIG. 1A depicts result demonstrating Luc expression in rats infected with ECTV-Luc and imaged 1 and 3 days post infection (dpi) using an IVS machine to measure light emission. FIG. 1B depicts results of antibodies for OPV (vaccinia virus; filled triangles) or Luc (open circles) in rats one month after ECTV-Luc infection or in uninfected naïve rats (closed squares). Rats were bled and antibodies were measured from the sera by enzyme-linked immunosorbent assay (ELISA).

FIG. 2 depicts exemplary results demonstrating OPV infection of human and rat tumor cells in vitro. Androgen-sensitive human prostate adenocarcinoma cells (LNCaP; left) and rat bladder urothelial carcinoma cells (AY-27; right) were infected with 10 plaque-forming units (pfu) of VACV (black bars) or ECTV (white bars) and viability was determined by trypan blue exclusion at 0, 1, 3 and 5 days post-infection.

FIG. 3 depicts exemplary results demonstrating that ECTV infects rat tumors in vivo and that it remains restricted to the tumor. A female Fischer 344 rat was injected with 5×10⁶ AY-27 tumor cells and, after 1 month, the tumor was injected with 1×10⁷ pfu of ECTV-Luc. Three days after infection, mice were imaged using an IVS machine to measure light emission after inoculating with luciferin.

FIG. 4 depicts exemplary results demonstrating the anti-tumor effect of intra-tumoral VACV infection in mice previously vaccinated against VACV. BALB/c mice (n=5) were either immunized with VACV (immunized-VV it) or not (Control and VV it). One month later, all mice were inoculated with 2×10⁵ mouse mammary adenocarcinoma (TS/A) tumor cells and, at 7 and 10 days after tumor challenge, 5×10⁶ pfu of VACV was injected intratumorally (VV it and immunized-VV it) or not (Control). Tumor volume was then monitored for a total of 18 days after tumor challenge.

FIG. 5 depicts a schematic of SARS-CoV-2 S protein-mediated infection and how neutralizing antibodies (Abs) may prevent it. In the receptor binding stage, the S1 subunit of SARS-CoV-2 binds ACE2 at the host cell surface. Neutralizing Abs can bind to the RBD domain on S1 to block the interaction of the RBD with ACE2. Crossreactive antibodies with other CoVs can bind conserved epitopes on the RBDs. After S1 is cleaved, the viral fusion peptide (FP) on the S2 subunit inserts into the host cell membrane, inducing the conformational change of the S2 subunit, which forms a six-helix bundle (6-HB) with HR1 and HR2 trimers. Antibodies that target the HR domains could block viral fusion.

FIG. 6 depicts exemplary results demonstrating detection of S and S1 expression in ECTV-S and ECTV-S1 by Western Blot. Lysates of BSC-1 cells infected with the indicated viruses were analyzed by Western Blot using anti-SARS-CoV-2 S1 Ab (Sino Biologicals). Molecular weight markers (MWM) are on the right with sizes (Kd) indicated.

FIG. 7 depicts exemplary results demonstrating that ECTV-S and ECTV-S1 induce strong anti-S Ab responses in mice. C57BL/6 mice (n=4) were left uninfected (filled circles) or infected with 3,000 pfu of ECTV-S (open squares), ECTV-S1 (shaded triangles) or ECTV-WT (filled squares) as a control. At 30 days post-infection (dpi), Abs to SARS-CoV-2 receptor-binding domain (RBD; right), S1 (middle) or S2 (left) were determined by ELISA using the indicated plate-immobilized proteins and anti-mouse IgG labeled with horseradish peroxidase as a secondary Ab.

FIG. 8 depicts a map of pBSSK ECTV7.5 EGFP (SEQ ID NO:4) used to produce ECTV-EGFP.

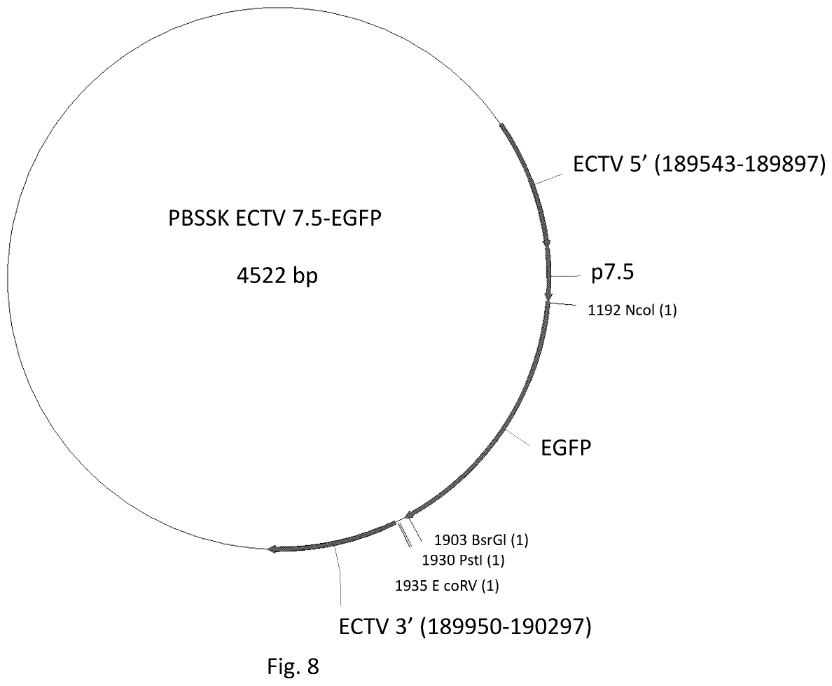

FIG. 9 depicts a map of pBSSK-ECTV036Rev (SEQ ID NO:5) used to produce novel ECTV recombinants by homologous recombination (selection of non-green plaques).

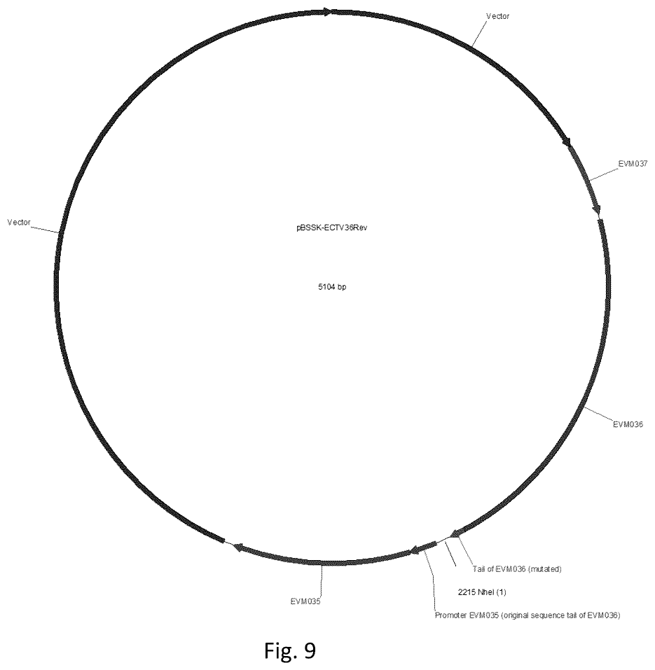

### DETAILED DESCRIPTION

In one embodiment, invention provides ectromelia virus (ECTV) vectors for use as expression vectors in vitro and in vivo. Whole genes, open reading frames (ORFs), and other exogenous nucleotide fragments, such as nucleic acid sequences to generate antibodies, antigens or antisense products, are contemplated for expression using the OPV vectors of the present invention.

Classes of genes contemplated for expression with the ECTV vectors of the present invention include tumor suppressor genes, cytotoxic genes, cytostatic genes, cytokines, and antigen encoding genes.

Also provided are methods of use of the ECTV vectors for treatment of diseases and disorders, including cancer and infectious disease. Such treatment includes methods of administering the ECTV vector of the invention comprising a heterologous nucleotide sequence for the treatment of the disease or disorder to a subject in need of treatment.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, “under transcriptional control” or “operably linked” refers to expression (e.g., transcription or translation) of a polynucleotide sequence which is controlled by an appropriate juxtaposition of an expression control element and a coding sequence. In one aspect, a DNA sequence is “operatively linked” or “operably linked” to an expression control sequence when the expression control sequence controls and regulates the transcription of that DNA sequence. A construct comprising a nucleic acid sequence operably linked to an expression control sequence is referred to herein as an “expression unit” or “expression cassette”.

As used herein, “an expression control sequence” refers to promoter sequences to bind RNA polymerase, enhancer sequences, respectively, and/or translation initiation sequences for ribosome binding. For example, a bacterial expression vector can include a promoter such as the lac promoter and for transcription initiation, the Shine-Dalgarno sequence and the start codon AUG. In some embodiments, a eukaryotic expression vector includes a heterologous, homologous, or chimeric promoter for RNA polymerase II, a downstream polyadenylation signal, the start codon AUG, and a termination codon for detachment of a ribosome.

As used herein, a “nucleic acid delivery vector” is a nucleic acid molecule which can transport a polynucleotide of interest into a cell. In one embodiment, such a vector comprises a coding sequence operably linked to an expression control sequence.

As used herein, “nucleic acid delivery,” or “nucleic acid transfer,” refers to the introduction of an exogenous polynucleotide (e.g., such as an expression cassette) into a host cell, irrespective of the method used for the introduction. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

As used herein, a “a recombinant vaccine vector” refers to a polynucleotide to be delivered into a host cell, either in vivo, ex vivo or in vitro which comprises genomic sequences from a vaccine virus and a heterologous nucleic acid sequence. In some embodiments, one or more virulence-associated sequences are inactivated in the vector. A vector may be encapsulated by viral capsid proteins or may comprise naked nucleic acids or may comprise nucleic acids associated with one or more molecules for facilitating entry into a cell (e.g., such as liposomes). Examples of vaccine viruses include, but are not limited to, poxviruses as further defined below.

As used herein, “an attenuated virus” or a virus having one or more “inactivated virulence associated genes” refers to a virus that is replication deficient or which replicates less efficiently than a wild type virus in a particular host.

As used herein, the term “administering a nucleic acid to a cell” or “administering a vector to a cell” refers to infecting (e.g., in the form of a virus), transducing, transfecting, microinjecting, electroporating, or shooting the cell with the nucleic acid/vector. In some aspects, molecules are introduced into a target cell by contacting the target cell with a delivery cell (e.g., by cell fusion or by lysing the delivery cell when it is in proximity to the target cell).

A cell has been “transformed”, “transduced”, or “transfected” by exogenous or heterologous nucleic acids when such nucleic acids have been introduced inside the cell. Transforming DNA may or may not be integrated (covalently linked) with chromosomal DNA making up the genome of the cell. In prokaryotes, yeast, and mammalian cells for example, the transforming DNA may be maintained on an episomal element, such as a plasmid. In a eukaryotic cell, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations (e.g., at least about 10).

As used herein, the term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart.

As used herein, a “target cell” or “recipient cell” refers to an individual cell or cell which is desired to be, or has been, a recipient of exogenous nucleic acid molecules, polynucleotides and/or proteins. The term is also intended to include progeny of a single cell, and the progeny may not necessarily be completely identical (in morphology or in genomic or total DNA complement) to the original parent cell due to natural, accidental, or deliberate mutation. A target cell may be in contact with other cells (e.g., as in a tissue) or may be found circulating within the body of an organism.

As used herein, a “subject” is a vertebrate, including a mammal. Mammals include, but are not limited to, murines, non-human primates, humans, farm animals, sport animals, pets, and feral or wild animals.

The terms “cancer,” “neoplasm,” and “tumor,” are used interchangeably and in either the singular or plural form, refer to cells that have undergone a malignant transformation that makes them pathological to the host organism. Primary cancer cells transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives.

The term “antigen source” as used herein covers any substance that will elicit an innate or adaptive immune response. An antigen source may require processing (e.g., such as proteolysis) to produce an antigen. An antigen source may be a polypeptide/protein, peptide, microorganism, tissue, oligo- or polysaccharide, nucleic acid (encoding an antigen or a polypeptide/protein comprising an antigen or itself serving as the antigen).

As used herein, the terms “antigen”, “antigenic determinant” or “epitope” are used synonymously to refer to a short peptide sequence or oligosaccharide, that is specifically recognized or specifically bound by a component of the immune system. Generally, antigens are recognized in the context of an MHC/HLA molecule to which they are bound on an antigen presenting cell.

As used herein, a “therapeutic vaccine” is a vaccine designed to boost the immune response to an antigen in a subject already exposed to the antigen.

As used herein, a “therapeutically effective amount” refers to an amount sufficient to prevent, correct and/or normalize an abnormal physiological response. In one aspect, a “therapeutically effective amount” is an amount sufficient to reduce by at least about 30 percent, by at least 50 percent, or by at least 90 percent, a clinically significant feature of pathology, such as for example, suppression of CD4 cells, decrease in viral load; decrease in size of a tumor mass, and the like. In one embodiment, a “therapeutically effective amount of a vaccine composition” enhances a beneficial immune response to a vaccine antigen by at least about 30%, by at least about 50%, or by at least about 90%, i.e., increasing CTL responses against the antigen, increasing secretion of γ-IFN by CD8+ T, increasing production of antibodies specific for a vaccine antigen or increasing the duration of these responses after administration of a vaccine composition.

As used herein, an immune response with “increased duration” refers to a significant response observed at least about 4 months, about 6 months, about 8 months, about 10 months, about 12 months, about 16 months, about 18 months, or at least about 20 months after initial administration of an antigen.

An “antibody” is any immunoglobulin, including antibodies and fragments thereof, that binds a specific antigen. The term encompasses polyclonal, monoclonal, and chimeric antibodies (e.g., bispecific antibodies). An “antibody combining site” is that structural portion of an antibody molecule comprised of heavy and light chain variable and hypervariable regions that specifically binds antigen. Exemplary antibody molecules are intact immunoglobulin molecules, substantially intact immunoglobulin molecules, and those portions of an immunoglobulin molecule that contains the paratope, including Fab, Fab′, F(ab′)2 and F(v) portions.

As used herein, the term “immune effector cells” refers to cells capable of binding an antigen and which mediate an immune response. These cells include, but are not limited to, T cells, B cells, monocytes, macrophages, dendritic cells, NK cells and cytotoxic T lymphocytes (CTLs), for example CTL lines, CTL clones, and CTLs from tumor, inflammatory, or other infiltrates.

As used herein, the term “viral infection” describes a disease state in which a virus invades healthy cells, uses the cell's reproductive machinery to multiply or replicate and ultimately lyse the cell resulting in cell death, release of viral particles and the infection of other cells by the newly produced progeny viruses. A “non-productive infection”, i.e., by a vaccine virus vector is an infection in which the vector is introduced into a cell but does not replicate within the cell, either because of inactivation of virulence associated gene(s) or because of a restricted host-range.

As used herein, the term “treating or preventing viral infections” means to inhibit the replication of the particular virus, to inhibit viral transmission, or to prevent the virus from establishing itself in its host, and to ameliorate or alleviate the symptoms of the disease caused by the viral infection.

As used herein, an “adjuvant” refers to a substance that enhances, augments or potentiates the host's immune response to a vaccine antigen.

The term “immunogenicity” means relative effectiveness of an immunogen or antigen to induce an immune response.

As used herein, a “booster” refers to a second or later vaccine dose given after the primary dose(s) to increase the immune response to the original vaccine antigen(s). The vaccine given as the booster dose may or may not be the same as the primary vaccine.

As used herein, “immunity” refers to natural or acquired resistance provided by the immune system to a specific disease. Immunity may be partial or complete, specific or nonspecific, long-lasting or temporary.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

In one embodiment, the present invention provides an Orthopoxvirus (OPV) vector in which a gene encoding an antigenic polypeptide is expressed from an early/late promoter. The large genome size of these viruses permits the engineering of vectors capable of accepting at least 25,000 base pairs of foreign DNA (Smith, et al., Gene 25: 21, 1983). Additionally, poxviruses can infect most eukaryotic cell types and do not require specific receptors for entry into a cell. Unlike other DNA viruses, poxviruses replicate exclusively in the cytoplasm of infected cells, reducing the possibility of genetic exchange of recombinant viral DNA with the host chromosome and allowing heterologous genes to be expressed independent of host cell regulation.

In some embodiments, the OPV vector may be an ectromelia virus (ECTV) vector. No serious case of infection of humans (adults) by ECTV has been reported.

The ECTV vectors of the invention include recombinant vectors comprising a heterologous nucleotide sequence under control of a viral early/late promoter. Methods and conditions for constructing recombinant poxvirus virus vectors, such as vaccinia virus vectors, are known in the art (see, e.g., Piccini, et al., Methods of Enzymology 153: 545-563, 1987; U.S. Pat. Nos. 4,769,330; 4,722,848; 4,769,330; 4,603,112; 5,110,587; 5,174,993; EP 83 286; EP 206 920; Mayr et al., Infection 3: 6-14, 1975; Sutter and Moss, Proc. Natl. Acad. Sci. USA 89: 10847-10851, 1992).

A vaccine vector is generally prepared as follows. In one aspect, a donor plasmid comprising a nucleic acid sequence encoding target nucleotide sequence is constructed, amplified by growth in a host cell and isolated by conventional procedures. The donor plasmid comprises a nucleic acid sequence homologous to vaccinia virus sequences. The nucleic acid encoding a target nucleotide sequence is operably linked to an expression control element. In one embodiment, the expression control element comprises viral regulatory elements, including upstream promoter sequences and, where necessary, RNA processing signals. The expression control sequences may be from a vaccinia virus, or other poxvirus, and is operably linked to the heterologous nucleotide sequence encoding the target sequence. The choice of promoter determines both the time (e.g., early or late) and level of expression of the target nucleotide sequence.

In some embodiments, the target nucleotide sequence is under the control of a viral early/late promoter. In one embodiment, the early/late promoter is a native, or wild-type, promoter from an OPV. In one embodiment, the early/late promoter is a synthetic promoter. Exemplary OPV early/late promotes that can be used include, but are not limited to, 7.5 and H5.

The expression unit comprising the expression control sequence and target nucleotide sequence is flanked on both ends by DNA homologous to a vaccinia virus DNA sequence being targeted as a recombination site. In some embodiments, the flanking sequences correspond to a nonessential locus in the viral genome. The resulting plasmid construct is then amplified by replication in E. coli or other suitable host and isolated using methods routine in the art (see, e.g., Maniatis, T., Fritsch, E. F., and Sambrook, J., In Molecular Cloning: A Laboratory Manual (Cold Spring Harbor Laboratory, Cold Spring Harbor, N.Y.) (1989)).

In some embodiments, a suitable cell culture (e.g., chicken embryo fibroblasts, CV-1 cells, BHK-21 cells, 143B tk-cells, vero cells, lung cells, etc.) is transfected with the donor plasmid along with recipient ectromelia virus sequences to select for recombinants that comprise both donor and recipient sequences. In certain instances, transfection may be facilitated by providing one or more molecules for facilitating entry of a nucleic acid into a cell. Suitable delivery vehicles include, but are not limited to: liposomal formulations, polypeptides; polysaccharides; lipopolysaccharides, cationic molecules, cell delivery vehicles, vehicles for facilitating electroporation, and the like.

In some embodiments, recipient sequences are selected which will result in the production of a recombinant virus that can induce and/or enhance a protective immune response and which lacks any significant pathogenic properties. Therefore, in one embodiment, the recipient sequence comprises one or more genes which are nonessential for growth of the virus in tissue culture and whose deletion or inactivation reduces virulence in a host organism, such as mammal (e.g., such as a mouse or a human being).

Inactivated virulence associated sequences can comprise whole or partially deleted gene sequences, substitutions, rearrangements, insertions, combinations thereof and the like. Mutations can be engineered or selected for. For example, an attenuated viral strain can be selected for by repeated passages in a suitable host cell and subsequent plaque purification can be used to identify plaques which are smaller, replicate more slowly, or which display other indications of complete or partial attenuation.

Host restricted viruses such as ECTV viruses can also be used as these are nonvirulent in some mammals, such as humans. In one embodiment, the ECTV vector is an attenuated ECTV, for example, in one embodiment, the attenuated ECTV virus vector is a vector in which the EVM036 protein, which is required for ECTV to spread from cell to cell in tissue culture, has been deleted or inactivated. In one embodiment, the Type I interferon (IFN-I) decoy receptor EVM166 has been deleted or inactivated.

Recombination between a homologous OPV virus sequence in the donor plasmid and the viral genome results in production of a recombinant OPV vector that comprises the target nucleotide sequence.

Recombinants can be detected by screening. In one embodiment, the screening method comprises a screen for plaque size, for example using ECTV-delta036 as an acceptor. In one embodiment, recombinants are screened by including reporter gene sequences in the donor plasmid and screening for recombinant viruses that carry these sequences. In one embodiment the reporter gene sequence encodes for a fluorescent reporter protein such as, but not limited to, green fluorescent protein (GFP) or dsRED. In one embodiment, the screening method is a color based screening assay. For example, donor plasmids that contain the E. coli β-galactosidase gene provide a method of distinguishing recombinant from parental viruses (Chakrabarti, et al., Mol. Cell. Biol. 5: 3403, 1985). Plaques formed by such recombinants can be positively identified by the blue color that forms upon addition of an appropriate indicator. Alternatively, or additionally, the recipient sequence comprises a reporter sequence and recombinants are detected by loss of function of the reporter sequence (i.e., resulting from insertion of donor sequences into the recipient sequence). In one aspect, the recipient reporter sequence is a virulence associated gene.

In some instances, viral particles can be recovered from the culture supernatant or from the cultured cells after a lysis step (e.g., chemical lysis, freezing/thawing, osmotic shock, sonication and the like). Consecutive rounds of plaque purification can be used to remove contaminating wild type virus. In some instances, viral particles can then be purified using the techniques known in the art (e.g., chromatographic methods or by ultracentrifugation on cesium chloride or sucrose gradients).

Vectors according to the invention may additionally comprise a detectable and/or selectable marker to verify that the vector has been successfully introduced in a target cell. These markers can encode an activity, such as, but not limited to, production of an RNA, peptide, or protein, or can provide a binding site for RNA, peptides, proteins, inorganic and organic compounds or compositions and the like. In some embodiments, the reporter sequence provided by the donor plasmid is used as the marker to verify introduction into a target cell.

Examples of detectable/selectable markers genes include, but are not limited to, nucleic acid sequences which encode products providing resistance to otherwise toxic compounds (e.g., such as antibiotics); products which are otherwise lacking in a recipient cell (e.g., tRNA genes, auxotrophic markers, and the like); products which suppress the activity of a gene product; enzymes (e.g., such as β-galactosidase or guanine-phosphoribosyl transferase), fluorescent proteins (GFP, CFP, YFG, BFP, RFP, EGFP, EYFP, EBFP, dsRed, mutated, modified, or enhanced forms thereof, and the like); cell surface proteins (i.e., which can be detected by an immunoassay); antisense oligonucleotides; and the like.

The marker gene can be used as a marker to confirm successful gene transfer by the vaccine vector and/or to isolate recombinants expressing the target nucleotide sequence.

In one embodiment, the vaccine vector comprises viral capsid molecules to facilitate entry of the vaccine vector into a cell. Additionally, viral capsid molecules may be engineered to include targeting moieties to facilitate targeting and/or selective entry into specific cell types. Suitable targeting molecules, include, but are not limited to: chemical conjugates, lipids, glycolipids, hormones, sugars, polymers (e.g. PEG, polylysine, PEI and the like), peptides, polypeptides, vitamins, lectins, antibodies and fragments thereof. In some embodiments, such targeting molecules recognize and bind to cell-specific markers of antigen presenting cells, such as dendritic cells (e.g., such as CD44) or cancer cells.

In one embodiment, a viral vector can be used for expression of two or more nucleotide sequences of interest. In one embodiment, two or more nucleic acid sequences or genes of interest are expressed from the same viral early/late promoter. In some embodiments, two or more nucleotide sequences or genes of interest are expressed from different viral promoters.

Nucleotide sequences that can be expressed using the viral vector of in the invention include, but are not limited to, a sequence encoding an RNA molecule (e.g., mRNA, siRNA, sgRNA, miRNA or shRNA), a sequence encoding a protein, a sequence encoding a peptide, a sequence encoding an antibody or fragment thereof, a sequence encoding a nanobody, a sequence encoding an antigenic polypeptide, and a sequence encoding a therapeutic agent.

In one embodiment, the target nucleic acid molecule is expressed under the control of a viral early/late promoter. As a result, the virus particle is taken up by a cell prior to expression of the target nucleic acid molecule, and the target nucleic acid molecule is transcribed and translated in the infected cell.

In one embodiment, the target nucleic acid molecule is expressed in the form of a fusion protein. In one embodiment, the target nucleic acid molecule is integrated into a viral gene such that the target nucleic acid molecule is linked to a gene encoding a marker or a viral protein. Such a vector can be constructed by a standard method using routine recombinant DNA technique.

In one embodiment, one or more genes that promote evasion of the host immune system are deleted or inactivation, resulting in a viral particle that has increased immunogenicity. Exemplary genes that promote evasion of the host immune system include, but are not limited to, those encoding secreted proteins which can act as either cytokine receptor homologues (viroceptors) or as cytokine mimics (virokines). Examples of viroceptors include the VACV secreted interleukin 1β (IL-1β) binding protein B15 and the interferon (IFN) type I binding protein B18. Virokines include, but are not limited to, the secreted VACV A39 smaphorin, which induces cytokine production from monocytes.

In one embodiment, the viral vector of the invention has been modified to carry at least one pro-apoptotic or pro-necroptotic gene. Exemplary pro-apoptotic or pro-necroptotic genes that can be included in the vector of the invention include, but are not limited to, CASP3, CASP9, APAF1, BAX, BAK1, BOK, BID, BCL2L11, BIM, BMF, BAD, BIK, HRK, PMAIP1, NOXA, BNIP3, BNIP3L, BCL2L14, BBC3, BCL2L12, BCL2L13, BCL-XS, RIPK1, RIPK3, MLKL, FAS, TRAIL1, TRAIL2 and TNFR-1.

In one embodiment of the present invention, the vector is an ECTV viral vector, or a fragment or variant thereof. In one embodiment, the ECTV viral vector comprises SEQ ID NO:1, or a fragment or variant thereof. In one embodiment, the ECTV viral vector comprises SEQ ID NO:1, or a fragment or variant thereof, and further comprises a coding sequence for expression of at least one heterologous sequence. An exemplary ECTV viral vector of the invention comprising a heterologous sequence for expression of EGFP is provided in SEQ ID NO:3, in which the coding sequence for EGFP is inserted at nucleotide position 189899 of SEQ ID NO: 1, resulting in the insertion of a coding sequence with an associated deletion of nucleotides 189899-189943 of SEQ ID NO:1. Therefore, in one embodiment, the composition comprises a fragment or variant of SEQ ID NO: 1 comprising about nucleotides 1-189898 and nucleotides 189944-209771 of SEQ ID NO: 1 and further comprising an insertion of a coding sequence for at least one heterologous sequence.

In one embodiment of the present invention, an EVM036 protein-defective viral vector is used, known as ECTV-Δ036. The EVM036 protein is a protein necessary ECTV to spread from cell to cell. Thus, this virus cannot constitute a virus particle having the ability to multiply autonomously after infection of cells of a subject and does not infect the other cells. The virus is therefore highly safe as a virus for vaccines. In one embodiment, the ECTV-Δ036 viral vector comprises SEQ ID NO:2, or a fragment or variant thereof. In one embodiment, the ECTV-Δ036 viral vector comprises SEQ ID NO:2, or a fragment or variant thereof, and further comprises a coding sequence for expression of at least one heterologous sequence. The ECTV-4036 viral vector provided in SEQ ID NO:2 comprises a deletion of nucleotides 49614-50731 of SEQ ID NO:1. Therefore, in one embodiment, the ECTV-Δ036 viral vector comprises a fragment or variant of SEQ ID NO: 1 comprising about nucleotides 1-49613, 50732-189898, and nucleotides 189944-209771 of SEQ ID NO: 1 and further comprising an insertion of a coding sequence for at least one heterologous sequence at about nucleotide position 189899 of SEQ ID NO:1.

In one embodiment of the present invention, an EVM166 protein-defective viral vector is used, known as ECTV-Δ166. EVM166 is a secreted decoy receptor that is able to bind Type-I interferons (IFNs) across several species, and promotes evasion of the host immune system in order to allow for survival and propagation of the virus.

### Antigenic Polypeptides

In one embodiment, the viral vector of the invention comprises a nucleotide sequence encoding an antigenic polypeptide. In one embodiment, the term “antigenic polypeptide” refers to a polypeptide that can induce or promote an immune response in a subject administered the virus vector of the present invention. For example, in certain embodiments, the antigen is associated with a cancer/tumor antigen, autoantigen (e.g., such as antigens recognized in transplant rejection); allergen; an antigen associated with hypersensitivity; prion antigen; viral antigen; a bacterial antigen, an antigen from protozoa or fungi; and a parasitic antigen.

### Viral Antigens

In certain embodiments, the antigen is a viral antigen, or fragment thereof, or variant thereof. For example, in some embodiments, the viral antigen is from a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. In some embodiments, the viral antigen is from human immunodeficiency virus (HIV), Chikungunya virus (CHIKV), dengue fever virus, papilloma viruses, for example, human papilloma virus (HPV), polio virus, hepatitis viruses, for example, hepatitis A virus (HAV), hepatitis B virus (HBV), hepatitis C virus (HCV), hepatitis D virus (HDV), and hepatitis E virus (HEV), smallpox virus (Variola major and minor), vaccinia virus, influenza virus, rhinoviruses, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), Middle Eastern respiratory virus (MERS), Middle Eastern respiratory virus (MERS), severe acute respiratory syndrome coronavirus (SARS), severe acute respiratory syndrome coronavirus 2 (SARS-CoV-2), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, lassa virus, arenavirus, or cancer causing virus. For example, in one embodiment, the antigenic polypeptide useful for use as a vaccine for SARS-CoV-2 infection is SARS-CoV-2 spike protein or a fragment thereof comprising the receptor binding domain (RBD).

### Bacterial Antigens

In certain embodiments, the antigen is a bacterial antigen or fragment or variant thereof. In some embodiments, the bacterial antigen is from a bacterium from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

The bacterium can be a gram positive bacterium or a gram negative bacterium. The bacterium can be an aerobic bacterium or an anerobic bacterium. The bacterium can be an autotrophic bacterium or a heterotrophic bacterium. The bacterium can be a mesophile, a neutrophile, an extremophile, an acidophile, an alkaliphile, a thermophile, a psychrophile, a halophile, or an osmophile.

The bacterium can be an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. The bacterium can be a mycobacteria, Clostridium tetani, Yersinia pestis, Bacillus anthracis, methicillin-resistant Staphylococcus aureus (MRSA), or Clostridium difficile.

### Parasitic Antigens

In certain embodiments, the antigen is a parasite antigen or fragment or variant thereof. In some embodiments, the parasite antigen is of a parasite from any one of a protozoa, helminth, or ectoparasite. The helminth (i.e., worm) can be a flatworm (e.g., flukes and tapeworms), a thorny-headed worm, or a round worm (e.g., pinworms). The ectoparasite can be lice, fleas, ticks, and mites.

The parasite can be any parasite causing any one of the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

The parasite can be Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus—lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

### Fungal Antigens

In certain embodiments, the antigen is a fungal antigen or fragment or variant thereof. The fungus can be Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

### Self Antigens

In some embodiments, the antigen is a self-antigen or a variant or fragment thereof. A self-antigen may be a constituent of the subject's own body that is capable of stimulating an immune response. In some embodiments, a self-antigen does not provoke an immune response unless the subject is in a disease state, e.g., an autoimmune disease.

In some embodiments, self-antigens may include, but are not limited to, cytokines, antibodies against viruses such as those listed above including HIV and Dengue, antigens affecting cancer progression or development, and cell surface receptors or transmembrane proteins.

In some embodiments, the antigen is a tumor antigen, such as a tumor-associated antigen (TAA) or tumor-specific antigen (TSA), or a variant or fragment thereof. Tumor antigens are proteins that are produced by tumor cells that elicit an immune response, particularly T-cell mediated immune responses. Tumor antigens are well known in the art and include, but are not limited to, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

Illustrative examples of a tumor associated surface antigen are CD10, CD19, CD20, CD22, CD33, Fms-like tyrosine kinase 3 (FLT-3, CD135), chondroitin sulfate proteoglycan 4 (CSPG4, melanoma-associated chondroitin sulfate proteoglycan), Epidermal growth factor receptor (EGFR), Her2neu, Her3, IGFR, CD133, IL3R, fibroblast activating protein (FAP), CDCP1, Derlin1, Tenascin, frizzled 1-10, the vascular antigens VEGFR2 (KDR/FLK1), VEGFR3 (FLT4, CD309), PDGFR-α (CD140a), PDGFR-.beta. (CD140b) Endoglin, CLEC14, Tem1-8, and Tie2. Further examples may include A33, CAMPATH-1 (CDw52), Carcinoembryonic antigen (CEA), Carboanhydrase IX (MN/CA IX), CD21, CD25, CD30, CD34, CD37, CD44v6, CD45, CD133, de2-7 EGFR, EGFRVIII, EpCAM, Ep-CAM, Folate-binding protein, G250, Fms-like tyrosine kinase 3 (FLT-3, CD135), c-Kit (CD117), CSF1R (CD115), HLA-DR, IGFR, IL-2 receptor, IL3R, MCSP (Melanoma-associated cell surface chondroitin sulphate proteoglycane), Muc-1, Prostate-specific membrane antigen (PSMA), Prostate stem cell antigen (PSCA), Prostate specific antigen (PSA), and TAG-72. Examples of antigens expressed on the extracellular matrix of tumors are tenascin and the fibroblast activating protein (FAP).

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

### Antibodies

In one embodiment, the viral expression vector of the invention can be used for expression of a synthetic antibody, a fragment thereof, or a variant thereof. The antibody, or fragment thereof, can bind or react with an antigen. In some embodiments, the antibody can treat, prevent, and/or protect against disease, such as an infection or cancer, in the subject administered a composition of the invention.

In some embodiments, the antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment, which comprises both antigen-binding sites. Accordingly, the antibody can be the Fab or F(ab′)2. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an ScFv antibody, a nanobody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

As described above, the antibody can be generated in the subject upon administration of the composition to the subject. The antibody may have a half-life within the subject. In some embodiments, the antibody may be modified to extend or shorten its half-life within the subject.

In one embodiment, the viral expression vector of the invention can be used for expression of a nucleotide sequence encoding a bispecific antibody, a fragment thereof, a variant thereof, or a combination thereof. The bispecific antibody can bind or react with two desired target molecules, including, but not limited to, an antigen, a ligand, a receptor, a ligand-receptor complex, and a marker (e.g., a cancer marker.)

### Methods

In one embodiment, the present invention provides a method for producing a modified OPV for expression of a target nucleic acid molecule from an early/late viral promoter. This method comprises the steps of: coculturing, with a cell, an OPV in which a target nucleic acid molecule is integrated in the viral vector under the control of an early/late promoter; and isolating a virus particle from the culture supernatant.

In one embodiment, the virus vector of the present invention has undergone nucleic acid inactivation treatment. The nucleic acid inactivation treatment refers to the inactivation of only the virus genome in the state where the three-dimensional structures of envelope proteins such as F protein and HN protein are maintained and these proteins have their functions. The nucleic acid inactivation treatment can be carried out by, for example, nucleic acid-alkylating agent treatment (e.g., β-propiolactone), hydrogen peroxide treatment, UV irradiation, exposure to radiation, or heat treatment. In one embodiment, the virus lacks the ability to multiply and thus cannot multiply in a recipient even if the live virus remains after the drug treatment; thus, the high safety of the inactivated vaccine can be kept.

### Methods of Vaccination

In one embodiment, the invention includes methods of inducing an immune response in a subject in need thereof comprising administering a viral vector of the invention, wherein the vector comprises a nucleotide sequence encoding an immunogenic protein or peptide. In one embodiment, a viral vector of the invention, wherein the vector comprises a nucleotide sequence for an immunogenic protein or peptide, serves a vaccine. Also provided herein is a method of treating, protecting against, and/or preventing disease in a subject in need thereof by administering the vaccine to the subject. Administration of the vaccine to the subject can induce or elicit an immune response in the subject. The induced immune response can be used to treat, prevent, and/or protect against disease, for example, cancer or an infectious disease, including but not limited to pathologies relating to SARS-CoV-2 infection. In one embodiment, the pathology relating to SARS-CoV-2 infection is COVID-19.

The induced immune response can be used to treat, prevent, and/or protect against cancer. The following are non-limiting examples of cancers that can be treated by the disclosed methods and compositions: acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocytoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

In one embodiment, the methods of the invention include administering a viral vector to a subject wherein the viral vector comprises an expression construct for expression of at least one antigenic protein or peptide, wherein the antigenic protein or peptide promotes the generation of an immune response against the encoded antigenic protein or peptide. In one embodiment, the methods of the invention include administering a viral vector to a subject wherein the viral vector comprises an expression construct for expression of at least one antigenic protein or peptide, wherein the antigenic protein or peptide promotes the generation of an immune response against an antigen that is not encoded. For example, in one embodiment, a viral vector of the invention encoding an antigen of a virus that the subject has previously been infected with or immunized against is administered to a subject to induce an immune response against a different disease or disorder or different infectious agent. In an exemplary embodiment, a viral vector of the invention encoding an antigen of a virus that the subject has previously been infected with or immunized against is administered to a tumor of a subject to induce an immune response in the tumor which can be harnessed for the treatment of the tumor.

In one embodiment, the methods of the invention include administering a viral vector to a subject wherein the viral vector comprises an expression construct for expression of two or more antigenic proteins or peptides, wherein the expression of the two or more antigenic proteins or peptides promotes the generation of an immune response against the encoded antigenic proteins or peptides. In some embodiments, two or more encoded antigenic proteins or peptides are peptides or proteins of the same infectious agent (e.g., the same virus). In some embodiments, two or more encoded antigenic proteins or peptides are peptides or proteins of different infectious agents (e.g., two or more different viruses or two or more different clades of the same virus). In some embodiments, two or more encoded antigenic proteins or peptides are peptides or proteins associated with the same disease or disorder (e.g., two or more antigenic proteins or peptides associated with the same cancer.) In some embodiments, two or more encoded antigenic proteins or peptides are peptides or proteins associated with different diseases or disorders. For example, in one embodiment, the viral vector comprises an expression construct encoding a first antigenic protein or peptide associated with a viral infection and a second antigenic protein or peptide associated with cancer (e.g., a combination of an influenza antigen and a cancer antigen or a combination of a human cytomegalovirus antigen and a cancer antigen.) In such an embodiment, the immune response induced against the encoded viral antigen is harnessed for use against the cancer associated with the encoded cancer antigen.

The induced immune response can include an induced humoral immune response and/or an induced cellular immune response. The humoral immune response can be induced by about 1.5-fold to about 16-fold, about 2-fold to about 12-fold, or about 3-fold to about 10-fold. The induced humoral immune response can include IgG antibodies and/or neutralizing antibodies. The induced cellular immune response can include a CD8⁺ T cell response, which is induced by about 2-fold to about 30-fold, about 3-fold to about 25-fold, or about 4-fold to about 20-fold.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

In one embodiment, the viral vector of the invention for expression of at least one antigenic polypeptide can be administered alone. In one embodiment, the viral vector of the invention for expression of at least one antigenic polypeptide can be administered in combination with another treatment for a disease or disorder.

In one embodiment the viral vector of the invention for expression of at least one antigenic polypeptide is administered in combination with an additional vaccine composition as a prime or a boost vaccine. In one embodiment, a subject who has been immunized with a vaccine (as a priming vaccine) is then administered a viral vector of the invention expressing at least one antigenic polypeptide as a boosting vaccine to increase the immune response.

In one embodiment the viral vector expresses at least two antigenic polypeptides, wherein at least one antigenic polypeptide is specific for a virus that a subject has been immunized against or for a common virus to which the subject likely has immunity. Therefore, in one embodiment, the viral vector of the invention is administered to a subject who has previously been immunized, wherein the viral vector comprises at least one antigenic polypeptide of a virus that the subject was immunized against and at least one additional antigenic polypeptide. In such an embodiment, the vaccine of the invention promotes an immune response against both the antigenic polypeptide of the virus that the subject was previously immunized against and the additional antigenic polypeptide.

### Administration

The compositions of the invention can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The composition can be administered prophylactically or therapeutically. In prophylactic administration, the compositions can be administered in an amount sufficient to induce an immune response. In therapeutic applications, the compositions are administered to a subject in need thereof in an amount sufficient to elicit a therapeutic effect. An amount adequate to accomplish this is defined as “therapeutically effective dose.” Amounts effective for this use will depend on, e.g., the particular composition of the treatment regimen administered, the manner of administration, the stage and severity of the disease, the general state of health of the patient, and the judgment of the prescribing physician.

The composition can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. One skilled in the art would know that the choice of a pharmaceutically acceptable carrier, including a physiologically acceptable compound, depends, for example, on the route of administration of the expression vector.

The composition can be delivered via a variety of routes. Typical delivery routes include parenteral administration, e.g., intradermal, intramuscular, intratumoral or subcutaneous delivery. Other routes include oral administration, intranasal, and intravaginal routes. For the DNA of the composition in particular, the composition can be delivered to the interstitial spaces of tissues of an individual (Felgner et al., U.S. Pat. Nos. 5,580,859 and 5,703,055, the contents of all of which are incorporated herein by reference in their entirety). The composition can also be administered to muscle, or can be administered via intradermal or subcutaneous injections, or transdermally, such as by iontophoresis. Epidermal administration of the composition can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

In one embodiment, the modified OPV of the present invention can be administered to cells of a mammal including a human.

In some embodiments, the viral vector of the present invention can be administered as an injection (subcutaneous, intradermal, or intramuscular injection) to cells of a mammal including a human. The injection can be prepared by a standard method. For example, a culture supernatant containing the virus vector is concentrated, if necessary, and suspended together with an appropriate carrier or excipient in a buffer solution such as PBS or saline. Then, the suspension can be sterilized by filtration through a filter or the like according to the need and subsequently charged into an aseptic container to prepare the injection. The injection may be supplemented with a stabilizer, a preservative, and the like, according to the need. The expression vector thus obtained can be administered as the injection to a subject.

In some embodiments, the viral vector can be formulated for administration by way of intradermal (ID) vaccination (e.g., ID injection by the Mantoux technique, use of a hollow microneedle, using a gene gun, using scarification or by other methods for ID delivery). The formulation for ID vaccination can be prepared by a standard method. For example, a culture supernatant containing the virus vector is concentrated, if necessary, and suspended together with an appropriate carrier or excipient in a buffer solution such as PBS, a virus vector-stabilizing solution, or saline. Then, the suspension can be sterilized by filtration through a filter or the like according to the need and subsequently charged into an aseptic container to prepare the formulation for ID vaccination. The formulation for ID vaccination may be supplemented with a stabilizer, a preservative, and the like, according to the need. The expression vector thus obtained can be administered intradermally to a subject.

The invention also provides a method for generating an immune response in an animal comprising administering any of the recombinant virus vectors or compositions described above to an animal in an amount effective to stimulate the immune response. In one embodiment, the immune response comprises one or more of the production of memory CD8+ T cells specific for an expressed target antigen, the production of memory CD4+ T cells specific for an expressed target antigen, and the production of antibodies specific for an expressed target antigen. In one embodiment, at least some of the antibodies are neutralizing antibodies.

In one embodiment, the animal is a human being. In one embodiment, the animal is a domestic animal such as a dog or cat. The animal may also be a feral or wild animal such as mink. The animal may also be a non-human primate.

The method may be used to provide a prophylactic or therapeutic composition to a patient at risk for being infected with or already infected with a viral agent, such as SARS-CoV-2. In one aspect, the method is used to provide a prophylactic vaccine to an individual at high risk of SARS-CoV-2 infection and the vaccine may be administered to an individual who is not SARS-CoV-2 positive at the time of first administration. However, the vaccine may also be administered to an individual who is SARS-CoV-2 positive at the time of first administration.

In one embodiment, the method may be used to provide an immunotherapeutic vaccine for the treatment of cancer, and thus induce an immune response against one or more cancer antigen. In one embodiment, the immune response comprises one or more of the production of memory CD8+ T cells specific for an expressed cancer antigen, the production of memory CD4+ T cells specific for an expressed cancer antigen, and the production of antibodies specific for an expressed cancer antigen.

The invention further provides pharmaceutical compositions (e.g., vaccines) comprising recombinant OPV vectors of the invention. In one embodiment, the composition comprises a pharmaceutically acceptable diluent, carrier, or excipient carrier. The composition may also contain an aqueous medium or a water-containing suspension, to increase the activity and/or the shelf life of the composition. The medium/suspension can include salt, glucose, pH buffers, stabilizers, emulsifiers, and preservatives.

In some embodiments, the composition further comprises an adjuvant, e.g., including, but not limited to: muramyl dipeptide; aluminum hydroxide; saponin; polyanions; anamphipatic substances; bacillus Calmette-Guerin (BCG); endotoxin lipopolysaccharides; keyhole limpet hemocyanin (GKLH); and cytoxan.

The invention also encompasses a kit including a OPV vector of the invention. The recombinant OPV vector can be provided in lyophilized form for reconstituting, for instance, in an isotonic aqueous, saline buffer. The kit can include a separate container containing a suitable carrier, diluent or excipient. The kit can also include one or more additional therapeutic agent, such as anti-cancer agents; agents for ameliorating symptoms of a viral infection (e.g., such as a protease inhibitor, Cimetidine (Smith/Kline, Pa.), low-dose cyclophospharide (Johnson/Mead, N.J.); and the like); and genes encoding proteins providing immune helper functions (such as B-7); and the like. Additionally, the kit can include instructions for mixing or combining ingredients and/or administering the kit components.

In one aspect, the invention provides a method of administering a therapeutically effective compositions according to the invention. The desired therapeutic effect comprises one or more of: reducing or eliminating viral load, increasing numbers of CD4+ and/or CD8+ T cells or antibodies which recognize the encoded antigen; increasing overall levels of CD4+ T cells; increasing levels of neutralizing antibodies which recognize the antigen; decreasing the number of or severity of symptoms of a disease; decreasing the expression of a cancer specific marker; decreasing size or rate of growth of a tumor; preventing metastasis of a tumor; preventing infection by a pathogenic organism; and the like. The therapeutic effect may be monitored by evaluating biological markers and/or abnormal physiological responses. Generally, an effective dose of a composition according to the invention comprises a titer that can modulate an immune response against the encoded antigen such that memory T cells are generated which are specific for the encoded antigen.

Both the dose and the administration means can be determined based on the condition of the patient (e.g., age, weight, general health), risk for developing a disease, or the state of progression of a disease.

In one embodiment, an effective amount of recombinant virus ranges from about 10 μl to about 25 μl of saline solution containing concentrations, of from about 1×10¹⁰ to 1×10¹¹ plaque forming units (pfa) virus/ml.

In one embodiment of the invention, a priming immunization is performed, followed, optionally, by a booster immunization at about 3-4 weeks after the priming immunization. However, subsequent immunizations need not be provided until at least about 4 months, about 6 months, about 8 months, about 12 months, about 10 months, about 16 months, about 18 months, or about 24 months after the priming boost. In one aspect, the composition is a prophylactic vaccine, administered to a patient who has not been exposed to the vaccine antigen, e.g., such as to an individual who is SARS-CoV-2 negative. In another aspect, the vaccine is administered therapeutically, to a person who is seropositive for the vaccine antigen (although not necessarily displaying symptoms) (i.e., such as to a SARS-CoV-2 positive individual).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Engineered ECTV Expresses Protein In Vivo and Induces an Immune Response

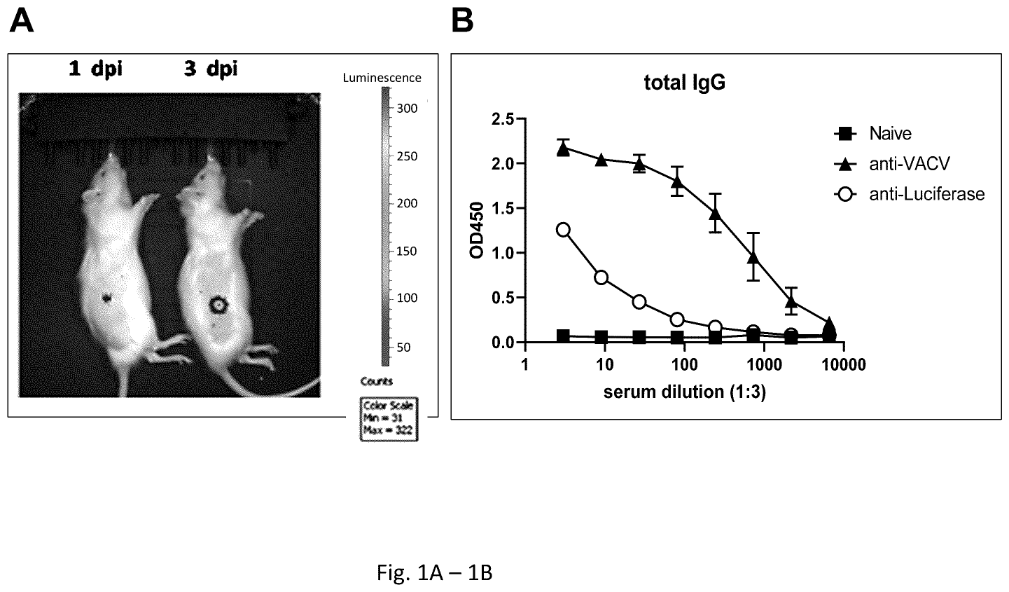

Orthopoxviruses (OPVs) use a large variety of proteins for cell entry, allowing them to infect a wide variety of cells (Moss et al., 2012, Viruses, 4(5):688-707). Thus, OPVs, including ECTV, can probably penetrate most mammalian cells. However, OPVs' capacity for productive infection is mediated by a large number of host-restriction genes that must be expressed in the infected cell (Oliveira et al., 2017, Viruses, 9(11):331). Consequently, the control of OPV in non-permissive hosts occurs after viral entry and before or soon after DNA replication. OPVs encode early genes that are transcribed from incoming virions before DNA replication, late genes that are transcribed after DNA replication, and early/late genes that are transcribed before and after viral replication (Meade et al., 2019, Wiley Interdiscip Rev RNA, 10(2): e1515). It is then plausible that ECTV in non-mouse cells can express early and early/late but not late genes. Without being bound by any particular scientific theory, it is hypothesized that proteins introduced into ECTV using an early/late promoter will be expressed from the incoming virions to induce immune responses but that the virus will not replicate in non-permissive hosts such as rats, hamsters, and humans. In support of this hypothesis, in rats inoculated intramuscularly with ECTV expressing firefly luciferase (ECTV-Luc) as a reporter, Luc activity was transiently observed at the infection site (FIG. 1A-B). While the virus did not spread, the rats mounted a robust antibody response to the virus and a weaker response to luciferase (FIG. 1B). These results indicate that ECTV is a suitable vector to induce immune responses in non-permissive species, such as humans. Importantly, due to non-permissivity, it is hypothesized that ECTV-based vaccines will likely be very safe. Moreover, if necessary, one could easily remove immune-evasion genes from ECTV to make it even safer and possibly more immunogenic (Xu et al., 2008, The Journal of experimental medicine, 205(4):981-92; Roscoe et al., 2012, Journal of virology, 86(24): 13501-7; Rubio et al., 2013, Cell host & microbe, 13(6):701-10; Remakus et al., 2018, Journal of immunology, 200(10):3347-52).

### Example 2: OPVs Infect Human and Rat Tumor Cells

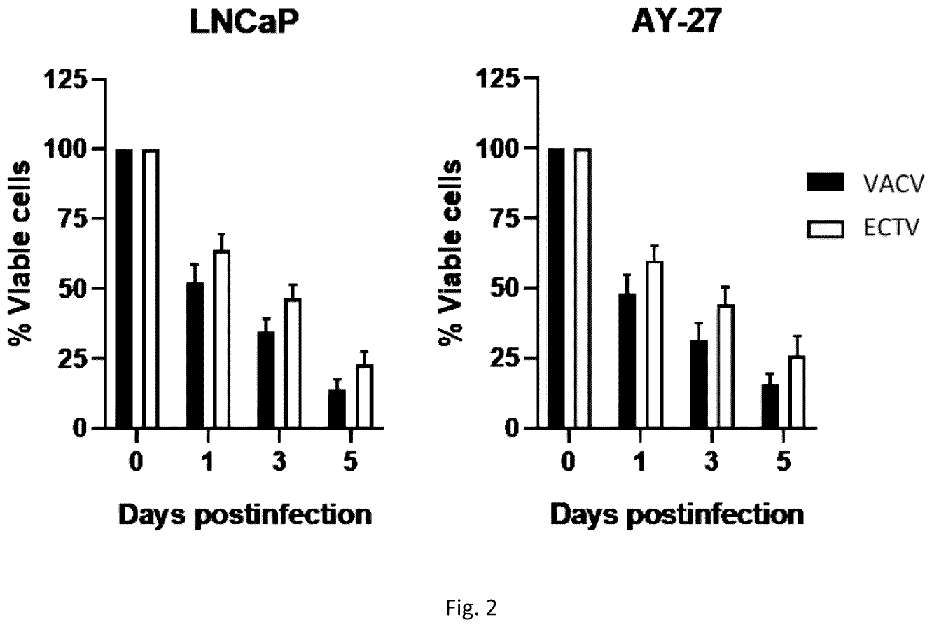

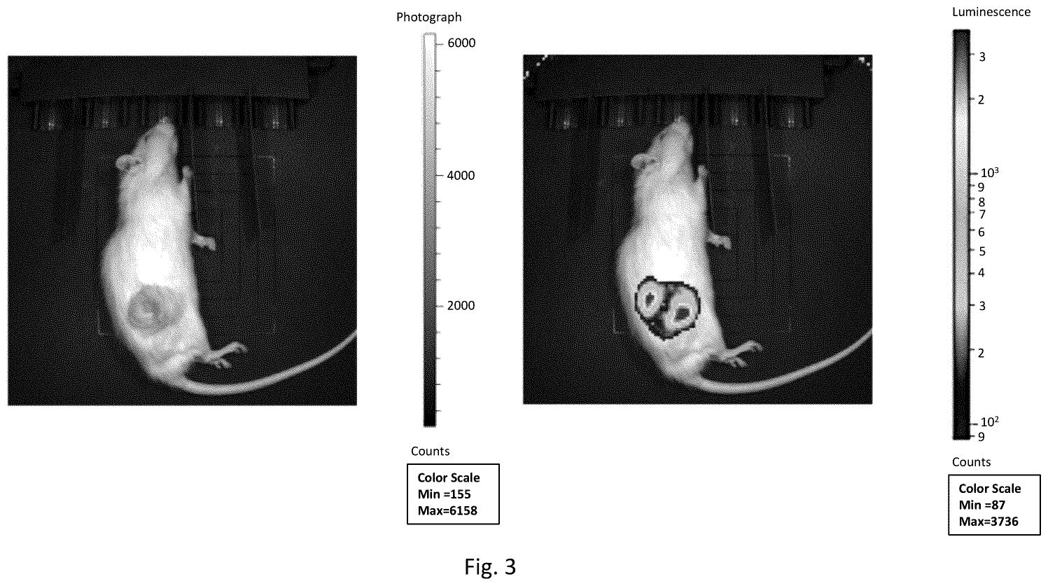

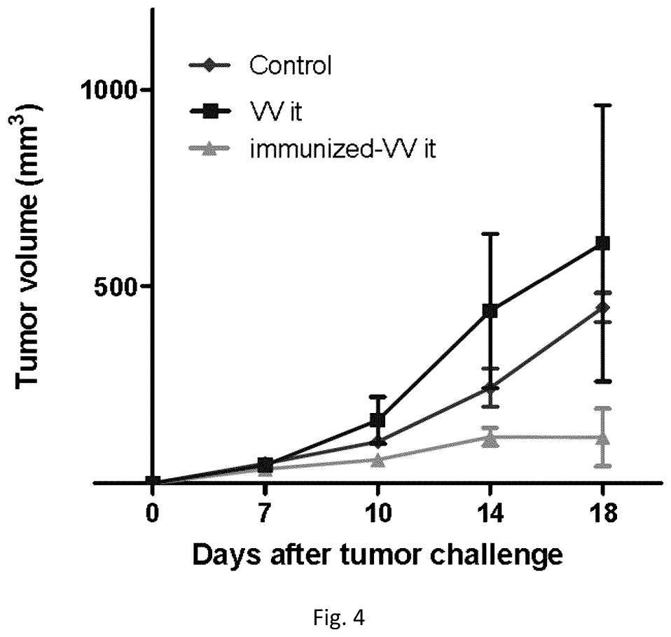

Despite their inability to systemically infect rats or humans, both VACV and ECTV were found to infect and lyse human (LNCaP) and rat (AY-27) tumor cells in vitro (FIG. 2). Further, luciferase produced by ECTV-Luc injected into rat AY-27 tumors remained localized to the site of the tumor, demonstrating the specificity of ECTV infection in vivo (FIG. 3). In addition, VACV injected into mouse mammary adenocarcinoma cell (TS/A) tumors of control and naïve (VV it) BALB/c mice had no significant ability to suppress tumor growth (FIG. 4). However, VACV injected into TS/A tumors of BALB/c mice that had previously been vaccinated against VACV (immunized-VV it) significantly inhibited tumor growth. Overall, these results suggest that OPVs, including ECTV and VACV, can prove useful as cancer immunotherapies to suppress tumor growth in vivo.

### Example 3: Combination Virus and Tumor Targeted Therapeutic

While the maximum insert size in ECTV has not been tested, VACV can accommodate at least 35 Kb. While not being bound by any particular scientific theory, it is therefore hypothesized that ECTV can accommodate similarly sized inserts.

Despite to its inability to infect other species, ECTV can infect human and rat tumor cell lines in vitro, and rat tumors in vivo without spreading to other tissues. Using the similar VACV, it was also found that rats pre-immune to VACV can eliminate tumors after intra-tumoral VACV infection. While not being bound by any particular scientific theory, it is hypothesized that this anti-tumor effect is not mediated by direct lysis of tumor cells but initially by anti-VACV CD8 T cells and likely continues through epitope spread to bona-fide tumor antigens. One might reasonably expect that the same will occur with ECTV in humans immunized with ECTV. Thus, while this approach could work with VACV, the use of ECTV is proposed as a much safer alternative. In addition, technologies have been developed to remove or add genes from ECTV. Thus, in addition to using wild type ECTV, patients may be administered ECTV engineered to express proteins of a virus for which they are already immune such as influenza A or human cytomegalovirus and already have CD8 T cells against. This would bypass the need to immunize against ECTV. In addition, one might engineer ECTV as an even more potent anti-tumor treatment. For example, one might use ECTV deleted of immune evasion genes to make it more immunogenic, or ECTV carrying pro-apoptosis or pro-necroptosis genes.

The use of ECTV is not limited to cancer immunotherapy but may also be effective in vaccinations against viral infection.

### Example 4: ECTV Expressing SARS-CoV Spike Protein is a Viable Vaccine Candidate

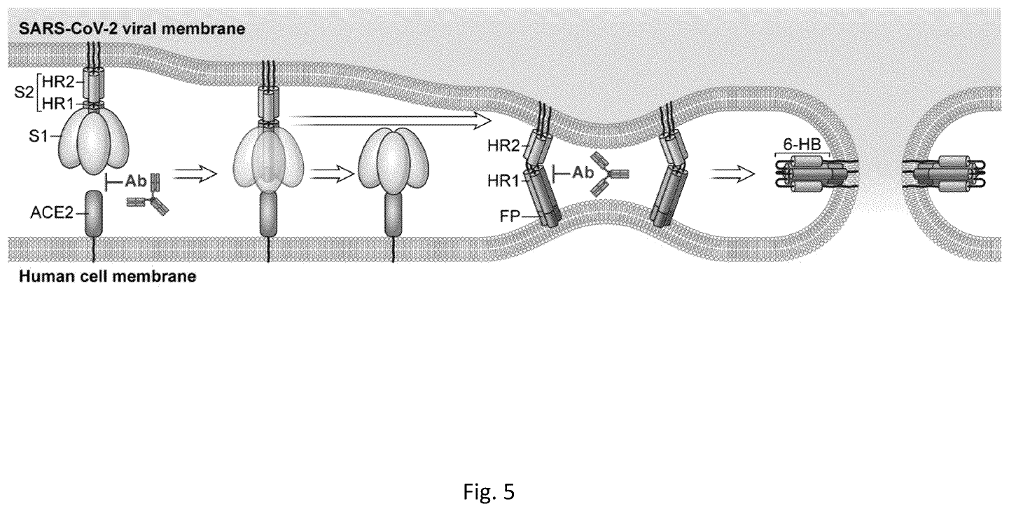

The entry of coronaviruses (CoVs) into target cells such as lung and gut epithelial cells (Hoffman et al., 2020, Cell, 181(2):271-280.e8) is mediated by Spike, a trimeric transmembrane viral protein present at the virion's surface. S is composed of S1 and S2 subunits. S1 contains a receptor-binding domain (RBD) whose function is to attach the target cell's virion through a cellular protein hijacked as a receptor. For SARS-CoV-1 and SARS-CoV-2, the cellular receptor is the transmembrane carboxypeptidase angiotensin-converting enzyme 2 (ACE2). S2 is necessary for the fusion of the viral envelope to the target cell membrane. For S2 to mediated fusion, S is first activated by proteolytic cleavage. In the case of SARS-CoV-2, it needs to be cleaved twice. The first cleavage, by the enzyme furin at a multi-basic S1/S2 site, occurs in the infected cell's secretory pathway. The second cleavage occurs on the surface of the target cell after S1 binds to ACE2. This cut is produced at the S2′ site by the Transmembrane serine protease 2 (TMPRSS2) and is critical for increasing the virus's infectivity. In cells that lack TMPRSS2, the second processing can be done in endosomes by cellular cathepsins but results in decreased infectivity (Hoffman et al., 2020, Cell, 181(2):271-280.e8; Bourgonje et al., 2020, J Pathol, 251(3):228-248; Xaio et al., 2020, Viruses, 12(5):491; Yan et al., 2020, Science, 367(6485): 1444-1448; Hoffmann et al., 2020, Molecular Cell, 78(4):779-84.e5; Ho et al., 2020, Antib Ther, 3(2): 109-14). Ideally, a vaccine to SARSCoV-2 should induce antibodies (Abs) capable of controlling not only SARS-CoV-2 but also other SARS-like CoVs that may emerge in the future. To achieve this type of vaccine, Abs must target epitopes that are conserved between similar CoVs such as SARS-CoV-1 and SARS-CoV-2. Most virus-neutralizing Abs isolated to date target conserved and non-conserved areas in the RBD of S1. Some neutralizing Abs that target conserved epitopes in S2 have also been isolated. Therefore, both S1 and S2 are potential targets of protective Abs (FIG. 5, adapted from (Ho et al., 2020, Antib Ther, 3(2): 109-14).

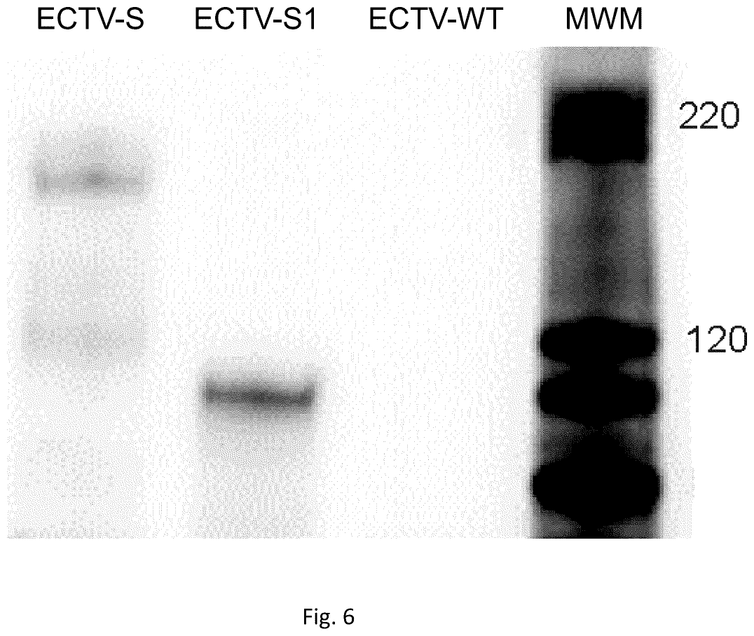

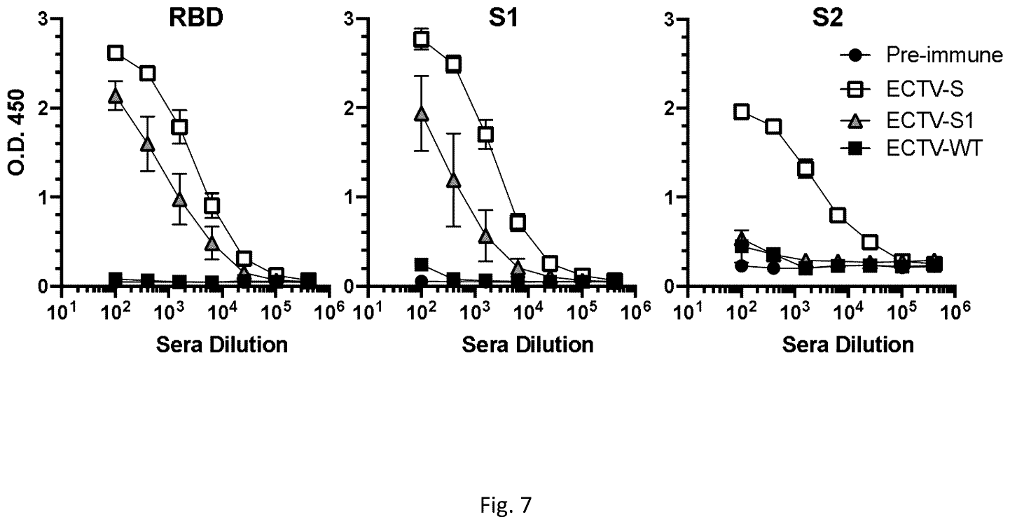

As an initial step to determine whether ECTV can be exploited as a vector for a COVID19 vaccine, homologous recombination was used to introduce human codon-optimized full-length S or the S1 subunits from SARS-CoV-2 driven by the potent early/late 7.5 promoter into ECTV to generate ECTV-S and ECTV-S1. Recombinant viruses were purified from single plaques. Inserts of the accurate size were amplified by PCR from viral DNA using specific primers. Sanger sequencing showed that the S and S1 DNA sequences in ECTV-S and ECTV-S1 were precise. Confirming proper expression of S and S1, Western Blot analysis of lysates of infected BS-C-1 cells probed with an anti-S1 Ab (Sino Biologicals) showed bands of the approximate expected sizes in ECTV-S (˜180 Kd) and ECTV-S1 (˜78 Kd). These bands were not present in lysates of cells infected with ECTV-WT (FIG. 6). Next, groups of 5 mice were bled and then infected with a single dose in the footpad of 3,000 pfu ECTV-WT, ECTV-S, or ECTV-S1. One month later, the mice were bled again, and Abs to S1, RBD, or S2 in preimmune and immune sera were determined by ELISA (FIG. 7). Results showed that sera from mice inoculated with ECTV-S and ECTV-S1, but not with ECTV-WT, contained high titer Abs to ECTV-S1 and RBD. Surprisingly, ECTV-S induced a more potent response to S1 and RBD than ECTV-S1. Also, only the sera from mice immunized with ECTV-S contained high Ab titers to S2. Together, the data indicate that ECTV isolates have been produced and warrant further testing as COVID19 vaccines.

### SEQUENCES

SEQ ID NO:1—Sequence of Ectromelia virus Moscow strain (ECTV-MOS). Virology. 2003; 317(1): 165-86), NCBI Reference Sequence: NC_004105.1.
SEQ ID NO:2—Sequence of ECTV-4036. Based on the ECTV-MOS sequence.
SEQ ID NO:3: Sequence of ECTV-EGFP. Based on the ECTV-MOS sequence.
SEQ ID NO:4 pBSSK ECTV7.5 EGFP
SEQ ID NO:5—pBSSK-ECTV036Rev

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A recombinant ectromelia virus (ECTV) vector comprising at least one expression unit for expression of at least one heterologous nucleic acid sequence.

2. The ECTV vector of claim 1 wherein the at least one expression unit is under the control of an ECTV early/late promoter.

3. The recombinant ECTV vector of claim 1, wherein the ECTV vector is attenuated.

4. The recombinant ECTV vector of claim 1, wherein the early/late promoter is selected from the group consisting of 7.5 and H5.

5. The recombinant ECTV vector of claim 1, wherein the ECTV vector comprises a deletion or inactivation of at least one immune evasion gene.

6. The recombinant ECTV vector of claim 5, wherein the at least one immune evasion gene is selected from the group consisting of a cytokine receptor homologue and a cytokine mimic.

7. The recombinant ECTV vector of claim 1, wherein the ECTV vector further comprises one or more additional heterologous nucleotide sequence.

8. The recombinant ECTV vector of claim 7, wherein the one or more additional heterologous nucleotide sequence is selected from the group consisting of a sequence encoding a therapeutic agent, a sequence encoding a targeting moiety, a sequence encoding a detectable and/or selectable marker, a pro-apoptotic gene, and a pro-necroptotic gene.

9. The recombinant ECTV vector of claim 1, wherein the target nucleotide sequence encodes a polypeptide selected from the group consisting of an antigenic polypeptide sequence, an antibody and an antibody fragment.

10. The recombinant ECTV vector of claim 9, wherein the antigenic polypeptide sequence is at least one selected from the group consisting of a cancer/tumor antigen, an autoantigen, an allergen, an antigen associated with hypersensitivity, a prion antigen, a viral antigen, a bacterial antigen, an antigen from protozoa or fungi, and a parasitic antigen.

11. The recombinant ECTV vector of claim 10, wherein the at least one antigen comprises a cancer specific antigen.

12. The recombinant ECTV vector of claim 10, wherein the at least one antigen comprises a viral antigen.

13. The recombinant ECTV vector of claim 12, wherein the viral antigen is selected from the group consisting of SARS-CoV-2 spike antigen, and a fragment of the SARS-CoV-2 spike antigen comprising the receptor binding domain (RBD).

14. The recombinant ECTV vector of claim 10, wherein the at least one antigen is a bacterial antigen.

15. The recombinant ECTV vector of claim 10, wherein the expression unit comprises at least two nucleotide sequences encoding antigenic polypeptides.

16. The recombinant ECTV vector of claim 15, wherein the at least two antigenic polypeptides are from two different viruses or from two different clades of the same virus.

17. The recombinant ECTV vector of claim 15, wherein the expression unit comprises at least one antigenic polypeptide from a virus and at least one cancer-specific antigenic polypeptide.

18. The recombinant ECTV vector of claim 15, wherein the at least one antigen is selected from the group consisting of a CTL-recognized epitope, a T helper cell-recognized epitope, and a B cell-recognized epitope.

19. The recombinant ECTV vector of any one of claims 1-18, wherein the vector comprises a nucleotide sequence selected from the group consisting of:
a) the nucleotide sequence of SEQ ID NO:1 further comprising a nucleotide sequence for expression of a heterologous sequence;
b) the nucleotide sequence of SEQ ID NO:2 further comprising a nucleotide sequence for expression of a heterologous sequence;
c) a fragment of the nucleotide sequence of SEQ ID NO: 1 further comprising a nucleotide sequence for expression of a heterologous sequence; and
d) a fragment of the nucleotide sequence of SEQ ID NO:2 further comprising a nucleotide sequence for expression of a heterologous sequence.

20. A vaccine composition comprising an ECTV vector.

21. The composition of claim 20, wherein the ECTV vector is a recombinant ECTV vector of any one of claims 1-19.

22. The composition of any one of claims 20-21, further comprising a pharmaceutical carrier.

23. A method for inducing an immune response in a subject comprising administering the vaccine composition of any one of claims 20-22 to the subject in an amount effective to induce an immune response.

24. The method of claim 23, wherein the immune response comprises one or more of: the production of memory CD8+ T cells specific for the at least one antigen, the production of memory CD4+ T cells specific for the at least one antigen, and the production of antibodies specific for the at least one antigen.

25. A method for treating cancer in a subject in need thereof, the method comprising administering the composition of any one of claims 20-22 to the subject.

26. The method of claim 25, wherein the recombinant ECTV vector comprises a target nucleotide sequence encoding a cancer antigen.

27. The method of claim 25, wherein the recombinant ECTV vector further comprises a target nucleotide sequence encoding a viral antigen.

28. The method of claim 27, wherein the viral antigen is selected from the group consisting of an influenza viral antigen and a human cytomegalovirus antigen.

29. The method of claim 25, wherein the recombinant ECTV vector comprises a target nucleotide sequence encoding an immunotherapeutic antibody for the treatment of cancer.

30. A method for treating a viral infection, or a disease or disorder associated therewith, in a subject in need thereof, the method comprising administering the composition of any one of claims 20-22 to the subject.

31. The method of claim 30, wherein the recombinant ECTV vector comprises a target nucleotide sequence encoding a viral antigen.

32. The method of claim 30, wherein the viral infection comprises SARS-CoV-2 infection.

33. The method of claim 30, wherein the recombinant ECTV vector comprises a target nucleotide sequence encodes an immunotherapeutic antibody for the treatment of the viral infection.
